(19) United States
(12) Patent Application Publication
(10) Pub. No.: US 2003/0013947 A1
(43) Pub. Date: Jan. 16, 2003

(54) Method and apparatus for non-invasive blood analyte detector

(76) Inventor: Joseph R. Frattarola, Landenberg, PA (US)

Correspondence Address: Pamela D. Politis, RatnerPrestia, Nemours Building, Suite 1100, P.O. Box 1596, Wilmington, DE, 19899, US

(21) Appl. No.: 10/191,310

(22) Filed: Jul. 9, 2002

Related U.S. Application Data
(60) Provisional application No. 60/304,085, filed on Jul. 10, 2001.

(51) Int. Cl. A61B 5/00

(52) U.S. Cl. 600/322

(57) ABSTRACT

A non-intrusive, self-calibrating and self-contained wearable device that measures the concentration of blood constituents or analytes is provided. The device employs low energy, short burst ultrasonic waveforms which are transmitted into the body and a resulting return echo waveform is electronically analyzed to determine a “real-time” blood constituent level. Two return waveform characteristics allow continuous automatic calibration for accurate blood measurements regardless of the patient's condition. The device is a completely self-contained system for the measurement, display and digital recording of blood glucose levels on a moment-to-moment basis.

### CROSS-REFERENCE TO RELATED APPLICATIONS

This application claims priority to U.S. Provisional Application No. 60/304,085, filed Jul. 10, 2001.

### FIELD OF THE INVENTION

This invention relates to the non-invasive sensing of blood analytes, more particularly, to non-invasive, self-calibrating blood glucose monitoring.

### BACKGROUND OF THE INVENTION

Determining the concentration of blood analytes of a subject is a critical aspect of diagnosing and treating many illnesses. Thus, the presence or amount of an analyte in a subject's blood or urine can provide information concerning the subject's drug use history, or a suitable therapeutic dosage. As a well-known example, the concentration of glucose in a subject's blood can provide useful information for management of hypoglycemia and hyperglycemia, particularly in diabetics.

Biochemical indicia can be measured in a blood sample from the subject. Where the subject's physiologic state may change significantly over short periods of time, such as blood glucose levels, samples for analysis may be taken more frequently. For some physiologic conditions the time scale for changes in the physiologic state can be short, so that removal and analysis of the appropriate sample at a preferred frequency is impractical. It is generally understood, for example, that more frequent sampling and analysis of a diabetic person's blood glucose, together with careful management of the person's sugar and insulin, can provide an improvement in quality of life and the lifespan of the diabetic. Removal of the blood sample, however, can be painful, and the apparatus surrounding the analysis of the sample is cumbersome and inconvenient to use.

For some types of biochemical indicia, there is a need for methods for “continual” monitoring, essentially measuring the biochemical indicia over extended monitoring time periods (for example, 24 hours per day throughout the week) substantially without interruption, or in a continuing series of measurements at appropriately spaced intervals.

More than 16 million people in the United States are afflicted with diabetes mellitus or have a predisposition to diabetes, and more than 750 thousand people are registered annually as diabetics. The medical complications associated with diabetes are quite serious, including increased risk of kidney, eye, nerve, and heart disease. To control their condition, diabetics must control their blood sugar levels by selecting proper nutrition and, in the more serious conditions, by administering insulin. To help guide their nutrition and insulin injections, diabetics must measure their sugar levels several times a day.

At present, all portable devices for measuring blood sugar require puncturing the fingertip to obtain a blood sample. The blood sample is then placed on a test strip that indicates the glucose concentration. An example is the ONE TOUCH® glucose meter sold by the LifeScan Co. These devices are very compact and reasonably accurate, but puncturing the fingertip to obtain a blood sample is inconvenient and painful and poses a risk of infection. No commercial alternatives are available.

A number of attempts have been made to measure blood sugar concentration noninvasively by measuring tissue absorption of light radiation in the near infrared energy spectrum—approximately 650 nm to 2700 nm. Difficulties arise using near IR because many wavelengths less than 2000 nm do not penetrate well through human skin. Devices applying multiple wavelengths of energy require complicated components, such as a continuous wide-band radiation source, which restricts the ability to construct a compact portable unit from these designs.

Ultrasound imaging is a critical tool in medicine. Ultrasound images provide detailed information because ultrasound frequencies are sensitive to subtle density differences found in various tissues. When ultrasonic waves are passed through a tissue, the waves are reflected in varying degrees based on the density and elasticity of the tissue. Despite the extensive use of ultrasound in diagnosing and treating disorders, ultrasound has not been employed to directly measure blood analytes.

Thus, there remains a need in the art for a device that continuously, instantaneously and accurately monitors blood glucose levels in a non-intrusive manner.

### SUMMARY OF THE INVENTION

The present invention relates to an apparatus adapted to be positioned adjacent to a subject tissue for the purpose of non-invasively measuring blood constituents is provided. The apparatus comprises an ultrasound transducer, preferably an integral power source coupled to the ultrasound transducer, and electronic circuitry connected to the ultrasound transducer. The electronic circuit provides data analysis so that ultrasonic frequency information is converted to a blood analyte concentration.

This apparatus may also comprise a contact pad contacting the ultrasound transducer and the subject tissue, a reflective plate to reflect ultrasonic waves, and a display component connected to the electronic circuit.

The present invention also provides a method of measuring a blood constituent. This method comprises emitting a calibration frequency signal into a subject tissue from a first position, detecting a calibration echo signal from a reflection of the calibration frequency signal, measuring a calibration transmittal time between emitting the calibration frequency signal and detecting the calibration echo signal, emitting a detection frequency signal with a first amplitude into the subject tissue from the first position, detecting an echo signal from the detection frequency signal and measuring a second amplitude of the detection frequency echo signal, measuring a detection transmittal time between emitting the detection frequency signal and is detecting the detection echo signal, and calculating a concentration of the blood constituent in the subject tissue from the calibration transmittal time, the detection transmittal time and an amplitude difference between the first and second detection frequency amplitudes.

### BRIEF DESCRIPTION OF THE DRAWINGS

FIG. 1 schematically illustrates a single transducer configuration of the present invention.

FIG. 2 schematically illustrates a dual transducer configuration of the present invention.

FIG. 3 schematically illustrates the face of an exemplary embodiment of the present invention configured as a monitor wearable on the wrist.

FIG. 4 schematically illustrates the back, or skin-facing side of the embodiment of the present invention shown in FIG. 3.

FIG. 5 schematically illustrates a side view of the embodiment of the present invention shown in FIG. 3.

FIGS. 6A-6D schematically illustrates exemplary transmitted and reflected ultrasonic signals of the present invention.

FIG. 7 is a flow chart of method steps according to one embodiment of the present invention.

FIG. 8 is an exemplary graph showing a relationship between blood glucose concentration and a detection signal amplitude change.

### DETAILED DESCRIPTION OF THE INVENTION

The invention will next be described with reference to the figures in which similar numerals indicate similar elements.

Ultrasound waves are high frequency sound waves, typically above 2 MHz, which is above the human audible range. Ultrasound transducers are used to transform alternating electrical currents into mechanical oscillations which form sound waves. Detecting and measuring ultrasonic waves are accomplished primarily through the use of a piezoelectric receiver or by optical means (i.e. crystals), because ultrasonic waves are rendered visible by the diffraction of light.

Ultrasonic frequency waves propagate through material as a function of the material properties. Some materials facilitate wave propagation, and ultrasonic waves transmit faster and experience less absorbency than may occur when the wave propagates through other media.

Within the same medium, the propagation properties of ultrasonic waves may vary as a function of frequency. Transmittal time and wave absorbency through a given medium may be frequency dependent. For example, higher frequency (i.e. greater than 10 MHz) ultrasonic waves are substantially insensitive to glucose concentration in a liquid medium, such that the propagation properties of high frequency ultrasonic waves remain significantly constant regardless of the glucose concentration in the liquid medium.

In contrast, lower frequency ultrasonic waves are sensitive to the glucose concentration of a liquid medium. Propagation properties of low frequency ultrasonic waves, such as transmittal time and absorbency, vary in a glucose concentration dependent manner. Because low frequency ultrasonic waves are glucose concentration dependent, ultrasonic frequency waves can be utilized to measure glucose concentrations in blood.

Furthermore, because both high and low ultrasonic frequency waves have similar propagation properties through body tissue, a combination of measurements using high and low frequency ultrasonic waves allows for a calibrated, non-invasive measurement of blood glucose concentration.

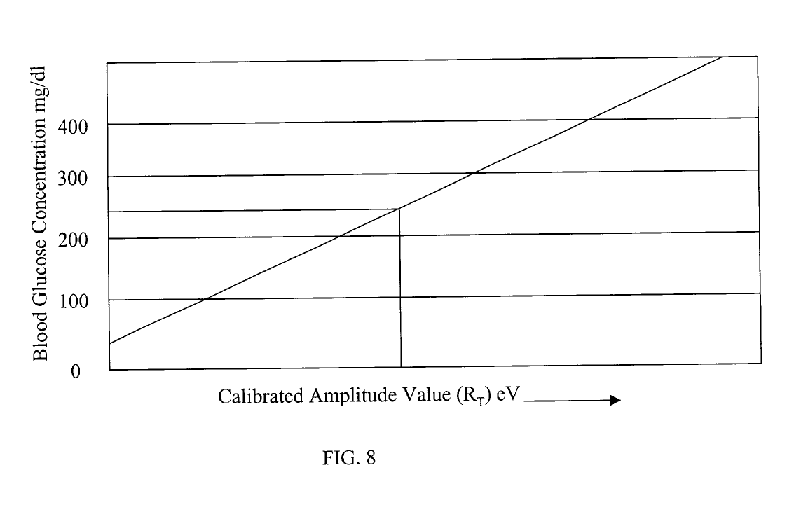

Although the detection ultrasonic frequency signal (lower frequency signal) is proportional to a subject's blood glucose concentration, the intensity of the signal for a given concentration will vary from person to person due to individual characteristics such as body fat, skin thickness, and blood vessel location. Consequently, to measure glucose concentration in absolute units (e.g., millimole/liter or milligram/deciliter), these individual characteristics must be taken into account for an accurate measurement. In a preferred embodiment, patients are given a glucose test during which various glucose concentrations are measured using both invasive methods (drawing blood) and the ultrasonic method of the present invention. This information is then used to calibrate the frequency readings so that measurements obtained by the ultrasonic method of the present invention can be converted to absolute units. For example a correlation chart, such as the one shown in FIG. 8, can 1o be generated for an individual and fed into a microprocessor. The microprocessor can then output the value of glucose concentration in absolute units, which can be shown on a display device of the apparatus.

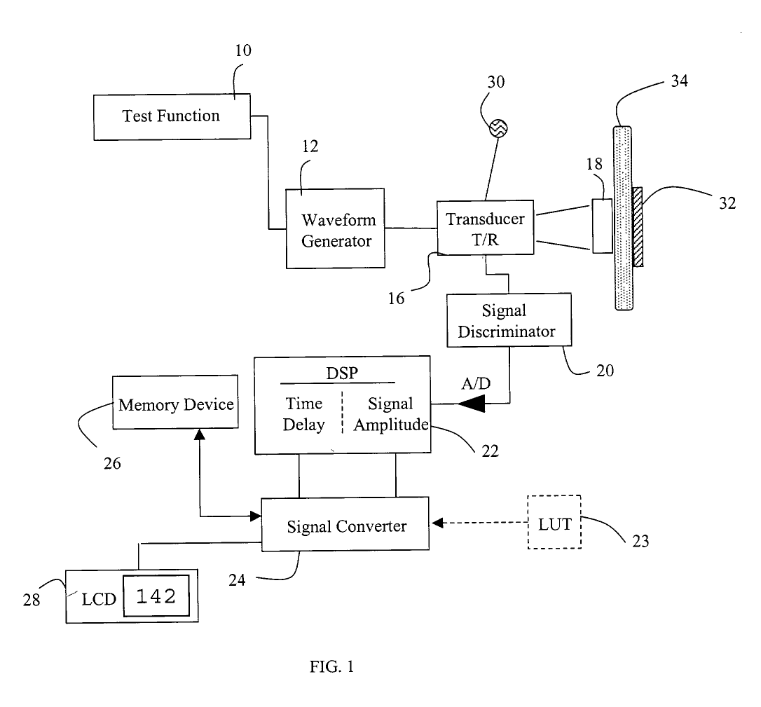

An exemplary embodiment of an apparatus according to this invention for non-invasive measurement of blood constituents or analytes is shown in FIG. 1.

This is an apparatus adapted to be positioned in contact with a subject tissue. The subject tissue is preferably a convenient part of the subject's body, such as an earlobe, wrist, or leg.

As shown in FIG. 1, the apparatus typically comprises an ultrasound transducer 16. FIG. 1 shows a single transducer serving both as transmitter and receiver, however separate transducers may be used for the transmit and receive functions. Preferably, the ultrasound transducer or ultrasonic wave transmitter provides ultrasonic frequencies between about 2 MHz to about 20 MHz, and is capable of transmitting at least two distinct frequencies within this range.

The ultrasound transducer is connected to a power source 30. The power source 30 may also be used to power other components of the system as necessary. The power source 30 may be self-contained, such as a battery, or an external source.

Associated with the ultrasound transducer 16 is electronic circuitry coupling various electronic components to generate, process, control and analyze ultrasonic frequency information from the transducer or transceiver, as needed. Ultimately, the electronic circuitry provides data analysis so that the ultrasonic frequency information is converted into a blood analyte concentration.

In the arrangement illustrated in FIG. 1, the transducer 16 is connected to a waveform generator 12. The waveform generator generates the wave component information which drives the transducer 16 to generate ultrasound frequency output. The waveform generator may itself receive wave function information from an external test function generator 10, or may contain an integral wave function generator. Associated with the transducer are signal detection and signal processing means. As shown in FIG. 1, the output of the transducer is provided to an ultrasonic signal discriminator 20. The signal discriminator 20 separates high ultrasonic frequency information from lower ultrasonic frequencies. The transducer output is typically an analog signal. This analog signal is converted to a digital signal for further processing. FIG. 1 shows the A/D conversion occurring prior to the discriminator circuit, however the A/D conversion may occur before or following the signal discriminator 20. Depending on the nature of the signal provided to the discriminator the discriminator 20 may be analog or digital.

The digital output from the discriminator is next processed in a digital signal processor (DSP) 22. Preferably the DSP has at least two channels for processing information. As shown in FIG. 1, the DSP comprises a channel for processing time information from the ultrasound transceiver, and a channel for processing signal amplitude information.

The DSP output is next supplied to a signal conversion circuit 24 for conversion of the raw digital output of the DSP into adjusted values. The signal converter 24 may be a simple calculating circuit able to apply preloaded predetermined correction factors to the raw data or may, preferably, include a programmed or programmable computer processing unit (CPU) which may be used to perform more complex processing of the raw data. The correction factors or other data needed to process the raw data from the transducer may be included in a Look Up Table 23 (LUT). The look-up table may be compiled as a calibrated set of data for each individual subject, or may be a generalized data set. If the look-up table contains generic conversion information, the device may be calibrated by the user for the individual subject. A generic conversion table may be provided which may be used by the user to calibrate the device for his individual use by running a calibration test as described earlier.

Still as shown in FIG. 1 associated with the signal converter 24 is, preferably, a memory device for data storage, which in a still more preferable embodiment comprises a removable recordable medium on which the data has been recorded.

The output of signal converter 24 typically is displayed on a liquid crystal display (LCD) 28, as well as being stored in the memory device 26. The memory device may store that display information, along with time and date information so that the blood analyte concentration information may be read and displayed in a time-dependent manner by the subject, or a health care practitioner. Such information may be helpful identifying blood analyte fluctuations and in managing disorders generally.

The electronic components associated with the transducer may, preferably, be contained in a microprocessor. Preferably, by using a central processing unit as part of the electronic components of this invention, the device may be readily customized to account for matching calibration factors and corrections, which are individual for each subject.

In addition to the electronic circuitry that processes the ultrasonic frequency signals, the apparatus may, instead of having the transducer contact the subject tissue directly, further comprise a contact pad 18 that contacts the ultrasound transducer and the subject tissue. The contact pad may be a conductive membrane or suave that facilitates ultrasonic frequency conduction from the ultrasonic transmitter to the subject tissue, and from the subject tissue to the ultrasonic receiver or transceiver.

The embodiment illustrated in FIG. 1 shows an apparatus in which the ultrasonic wave receiver is located in substantially the same position as the ultrasonic wave transmitter. In this configuration, the ultrasonic wave receiver detects an echo ultrasonic wave. In such case it is preferable that the device includes a reflective pad 32. The reflective surface is preferably highly reflective of ultrasonic frequency waves. Convenient and effective reflective surfaces include metallic plates.

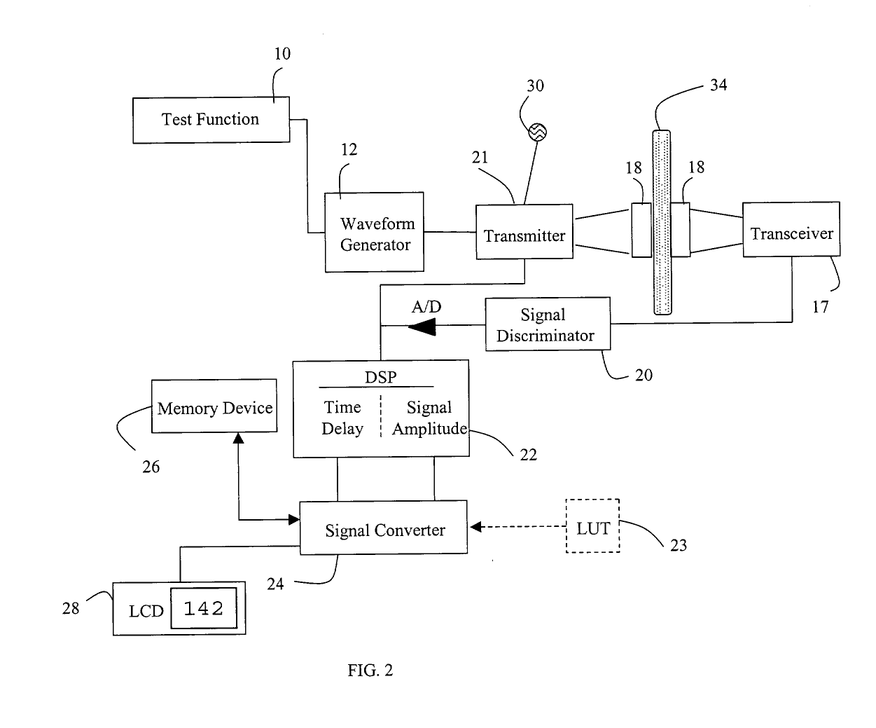

In an alternate embodiment shown in FIG. 2, the transducer comprises an ultrasonic wave transmitter 21 opposite an ultrasonic wave transceiver 17, with the subject tissue 34 positioned between the ultrasonic wave transmitter 21 and receiver 17. Preferably, contact pads 18 buffer the interface between the subject tissue and the transmitter and transceiver.

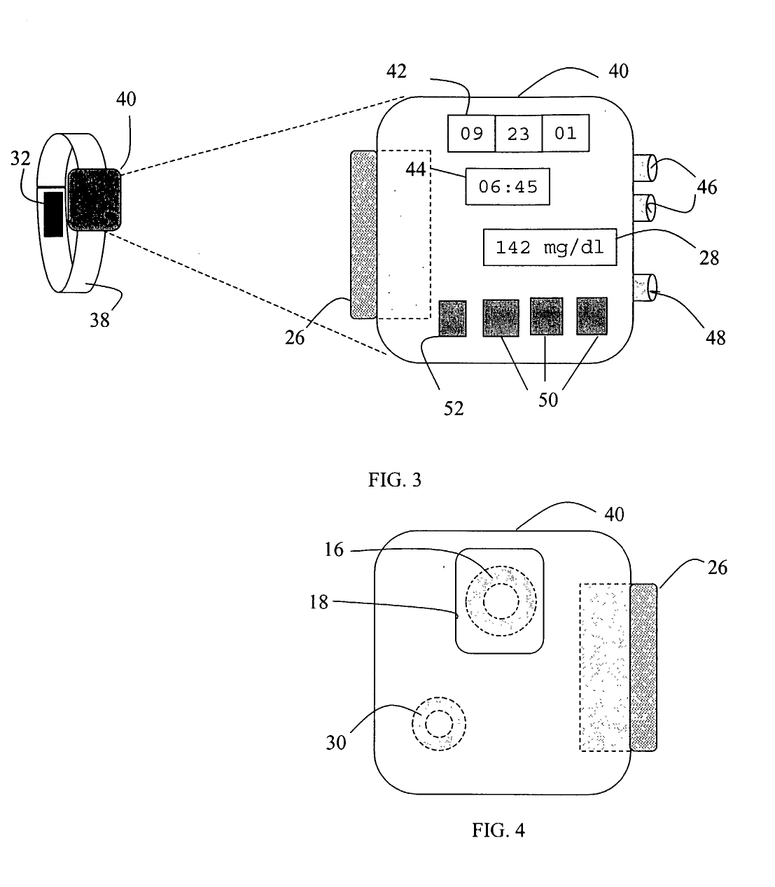

In one embodiment of the present invention, the apparatus is adapted to be positioned on the subject's wrist, much like a wristwatch. Such apparatus is shown in schematic representation in FIGS. 3,4 and 5. As shown in FIGS. 3, 4 and 5 the ultrasonic wave transmitter and receiver are a single ultrasonic transducer 16, which is positioned opposite reflective surface 32, with the subject tissue 34 being positioned between the ultrasound transducer 16 and the reflective surface 32. The electronic circuitry 36 is contained within the device 40. The electronic circuitry is capable of driving the transducer 16, processing the data and calculating a measurement reading for display. When using a wristwatch type embodiment, the data may be displayed in a liquid crystal display 40 as illustrated in FIG. 3. The display may show time 44 and date 42 as well as constituent or analyte measurements 28. An apparatus according to the present invention may also use dosage information to calculate an insulin dose, or dose of other treatment, according to the measurement acquired. Information such as insulin type 52 and dosage 50 may also be displayed. Such a device may have setting is controls 46 through which a measurement schedule may be set by the user. A manual test control 48 may also be available to initiate a measurement on demand.

In addition to the wrist configuration shown, other embodiments of the apparatus of the present invention may be adapted to be positioned on an earlobe, arm, leg, finger or other convenient body part.

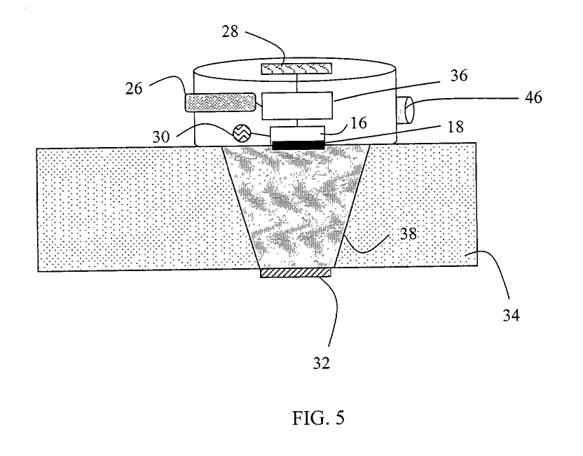

Regardless of the body part used for the subject tissue, continuous monitoring will be facilitated with an attachment device that attaches the apparatus to the subject. The wrist strap 38 shown in FIGS. 3 and 5 is but one such example.

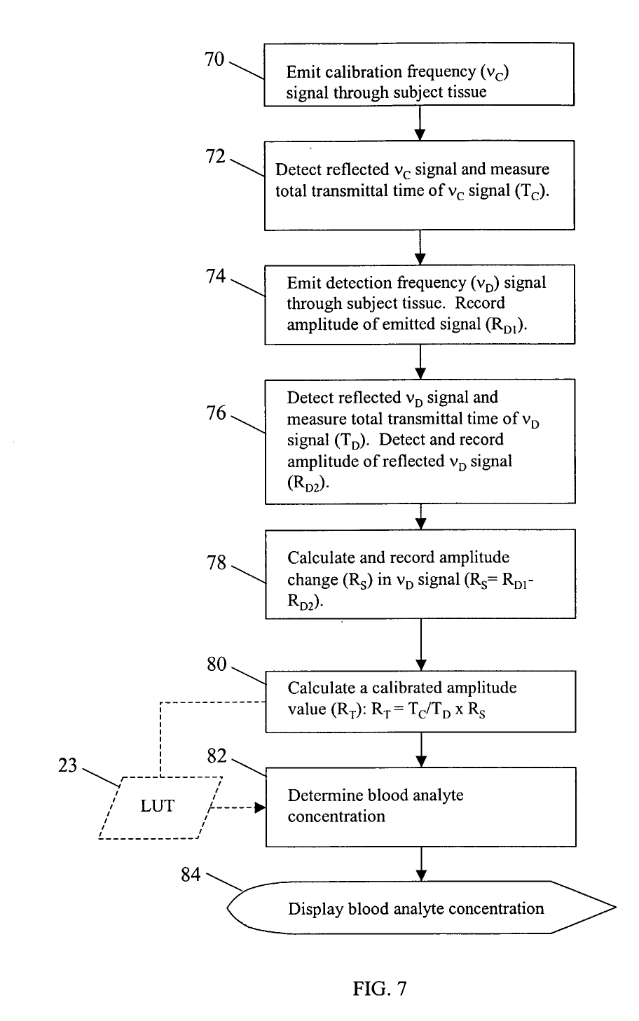

Operation of the apparatus according to this invention in measuring blood constituents or analytes implements the process illustrated as a flow chart in FIG. 7. This process comprises emitting a calibration frequency (v_{c}) signal into a subject tissue 70 from a first position adjacent to and coupled with the subject tissue, such as on a wrist. A calibration echo signal from a reflection of the calibration frequency signal is detected and the transmittal time of the v_{c} signal is measured 72 as T_{C}. (In the alternate embodiment illustrated in FIG. 2, the calibration signal transmittal time may also be measured from a second position, such as opposite the ultrasonic wave transmitter.). The measurement of the calibration transmittal time is the time between emitting the calibration frequency signal and detecting the calibration echo signal, or detecting the calibration signal from a second position.

A detection frequency (v_{D}) signal with a first amplitude R_{D1} is also emitted into the subject tissue from the first position 74. The detection transmittal time T_{D} and the amplitude of the echo signal from this detection frequency signal is measured 76. The echo signal of the detection frequency, or the signal after transmission through the subject tissue, has a second amplitude R_{D2}. The measurement of the detection transmittal time is the time between emitting the detection frequency signal and detecting the detection echo signal, or detecting the detection signal from a second position. Similarly, the amplitude of the detection frequency echo signal is measured as it is transmitted and received, regardless if it is transmitted and received at the same position.

From this information, a concentration of the blood constituent in the subject tissue is calculated from the calibration transmittal time T_{C}, the detection transmittal time T_{D} and an amplitude difference between the first and second detection frequency amplitudes as described in more detail below. The detection signal amplitude change R_{S} is calculated as R_{D1}-R_{D2} and recorded 78. A calibrated detection signal amplitude value is calculated using the time information that was collected. A correction factor ratio is applied to R_{S} to arrive at a calibrated amplitude value R_{T}, as shown in step 80. The calibrated amplitude value is used to determine the concentration of the desired blood analyte 82. The determination of the blood analyte concentration may be accomplished by accessing a look-up-table 23 that converts the calibrated amplitude value R_{T} to a blood analyte concentration value, which then is outputted to a display 84, and optionally a recording device.

LUT information that may be used to convert the calibrated amplitude value R_{T} to a blood analyte concentration value is typically provided by developing experimentally a calibration curve such as shown in FIG. 8. In this example, the blood analyte being measured is glucose, and FIG. 8 represents the glucose concentration as a function of measured ultrasonic frequency signal absorbency.

The calibration frequency signal and the detection frequency signal are both ultrasonic frequency signals, generally between about 2 MHz and about 20 MHz. To exploit the frequency dependent propagation behavior in measuring selected blood analytes, the calibration frequency is higher than the detection frequency. Preferably, the two ultrasonic frequencies are selected such that the measurement frequency to detect the raw blood glucose level is selected from the lower end of the ultrasound band (2 MHz to 3 MHz), and the calibration frequency used to determine the body character is selected from higher ultrasound frequencies (greater than about 6 MHz).

In one embodiment of the invention, a blood glucose measurement is conducted by transmitting a series of two ultrasound signals sequentially into the body. The first signal preferably comprises an ultrasonic frequency signal (v_{c}) at the high end of the ultrasound frequency band. A high frequency (v_{c}) is less vulnerable to interference when transmitted through the body. Such interference impacts the wave propagation velocity. A small reflective plate is positioned opposite the transceiver to provide a stable target for the waveform. The transmitted ultrasonic signal impinges the reflective target on the opposite side of the body and is reflected back through the body to the transceiver. This reflected signal, the echo signal, is received by the transceiver or transducer. The v_{c} signal's propagation time (T_{C}) is measured on a submicrosecond time base and stored for later use as a factor in calibrating the device.

Since this calibration frequency signal has preferably been selected as less disposed to velocity changes induced by variable biological conditions, the following relation provides an accurate determination of the signal velocity as it travels the distance between the transducer and reflective target and the return path to transducer:

V_{C} (propagation velocity of waveform through the body)=D_{C} (Total distance to and from reflector plate)/T_{C} (Time of waveform propagation).

A second ultrasound signal is also transmitted by the transducer to the reflective target, and the echo signal detected. The second signal preferably comprises a frequency signal at the lower end of the ultrasound frequency band (v_{D}), such that the signal, as it propagates through the body, is affected by variable biological conditions. The second signal may be transmitted into the body at any time relative to the first signal, as long as there is a sufficient time delay to prevent interference between the pulses. In practice, approximately one second between signal transmission is sufficient. Two components of the low frequency detection signal are determined: signal strength and time propagation delay. The return echo of the low frequency signal, as returned from the reflector plate, is electronically processed in the discriminator 20 to determine its signal amplitude change (R_{S}) and propagation time (T_{D}). The signal amplitude change R_{S} is the difference between the emitted signal amplitude (R_{D1}) and the amplitude of the return or detected signal (R_{D2}).

Return signal amplitude change (R_{S}) is proportional to the conductivity of the blood which is directly related to the amount of glucose in the blood. The amplitude change provides an indication of the raw blood glucose level. The low frequency signal experiences a travel time propagation delay period (T_{D}) due to interference with the body. This interference represents electrical characteristics of the body which provides a unique signature for the particular body and its current biological condition. By detecting this propagation time delay, a calibration factor may be 1o derived from the ratio of T_{C} and T_{D} which is applied to the detected signal amplitude change (R_{S}), to produce an accurate representation of a blood glucose reading regardless of patient or patient body condition. This compensating correction action is preformed on each test. The following relationships allow the calibration (high frequency) signal to provide a correlation ratio that is applied to the measurement low frequency signal strength information to yield a value that is easily converted to a true blood glucose value R_{T}.

V_{C}=D_{C}/T_{C}

V_{D}=D_{D}/T_{D}

Since: D_{C}=D_{D}, then V_{D}=(V_{C}×T_{C})/T_{D} and V_{D}/V_{C}=T_{C}/T_{D}.

Since the distance is constant, the result is a function of propagation time. The ratio of the measured values T_{C} and T_{D} is used to calculate the true blood glucose level in conjunction with the raw signal amplitude reading derived from R_{S}.

R_{T}=R_{S}(T_{C}/T_{D}).

The true glucose value is determined after both the measurement of change in signal amplitude (R_{S}) and propagation time values (T_{D}) are passed to the CPU 24. Stored body characteristic data is applied by the CPU to convert the calibrated signal strength measurement to a blood glucose value using calibration data to determine a true blood glucose level. This result is recorded in the memory device and sent to the LCD driver for visual display.

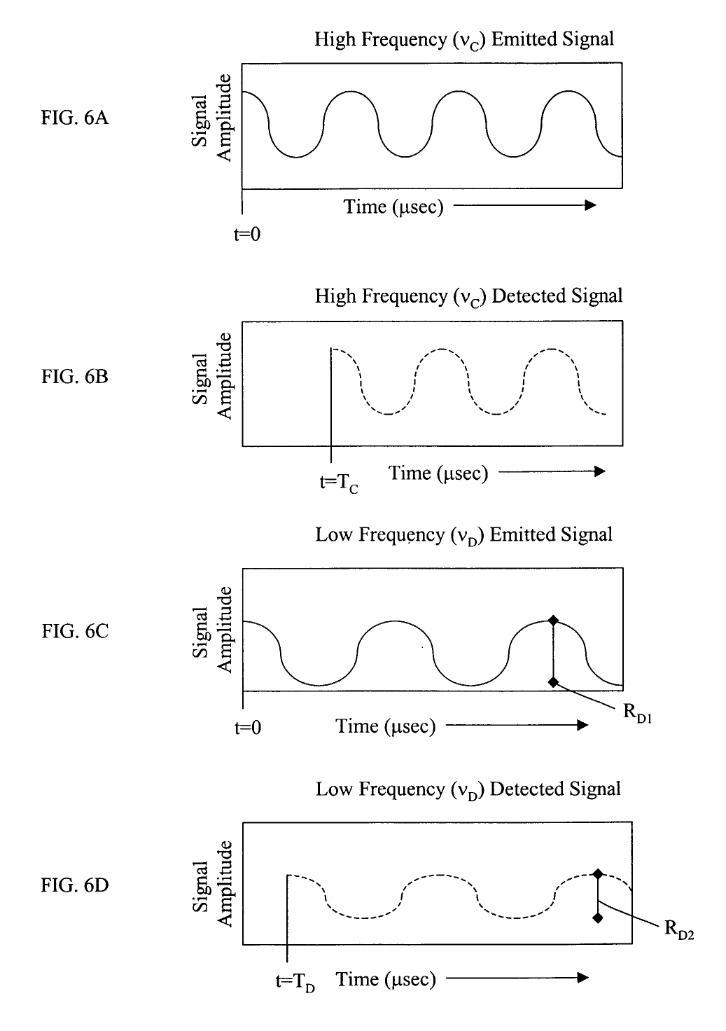

Schematic representations of example ultrasonic frequency waves are shown in FIGS. 6A-6D to illustrate the transmittal time and amplitude measurements of the present invention. FIG. 6A illustrates the amplitude of an emitted high frequency (v_{c}) ultrasonic wave as a function of time. FIG. 6B illustrates the same high frequency ultrasonic wave that is subsequently detected. The wave may be detected as an echo signal at the same location from which it was emitted, or a second position opposite the subject tissue from the emission location. The time delay between emitting the high frequency wave, and detecting the wave, or its echo, is T_{C}. As shown in FIGS. 6A and 6B, the amplitude of the high frequency calibration ultrasonic wave is substantially unmodified between the emission and detection points.

An example of a low frequency, or detection ultrasonic wave (V_{D}) is shown in FIGS. 6C and 6D. Because low frequency waves propagate faster as a function of glucose concentration, the time delay measurement between the emitted signal and the detection of the signal (T_{D}) is less than the time delay for the high frequency calibration wave in a medium containing dissolved glucose.

Furthermore, the amplitude of the low frequency ultrasonic wave is also sensitive to glucose concentration. As such, the amplitude of the emitted low frequency signal R_{D1} is greater than the amplitude of the detected low frequency signal amplitude R_{D2}.

This sensitivity of low frequency ultrasonic waveforms to blood glucose levels is shown schematically in FIGS. 6C and 6D. The originating input signals comprise a pulse of ultrasonic frequency having a signal strength of R_{D1}. The return echo signal is modified by the body in two aspects: its signal strength and body characteristic propagation time. The echo signal strength (R_{D2}) is a function of the amount of glucose in the body and the time delays (T_{C} and T_{D}) of the return signals defines the body characteristic correction factor. The amplitude of the low frequency waveform is diminished as a function of the glucose levels in the blood. The propagation time is delayed as a function of a collection of indices related to the body character.

The calibration frequency and the detection frequency signals may be reflected off a reflective surface prior to measuring the transmittal time and signal strength.

According to the present invention, specific blood analytes or constituents may be detected. The ultrasonic frequencies employed may be optimized to the specific analyte or constituent of interest. Analytes and constituents include glucose, cholesterol, sodium, hormones, pharmaceutical and illegal drug compounds. In one preferred embodiment of the invention, the blood constituent measured is glucose. Measurement of the blood constituent may be from a subject tissue such as an earlobe, an arm, a leg or a finger.

What is claimed:

1. An apparatus adapted to be positioned in contact with a subject tissue for the purpose of non-invasively measuring blood constituents, the apparatus comprising:
a) an ultrasound transducer for transmitting and receiving an ultrasound signal; and
b) electronic circuitry connected to the ultrasound transducer, for receiving a signal transmitted by said transducer through said subject tissue wherein said signal is modified through said transmission through said tissue, and for converting said received signal to a blood analyte concentration.

2. The apparatus according to claim 1 wherein said ultrasound transducer transmits a calibration ultrasound signal and a detection ultrasound signal and said electronic circuitry receives both signals and converts said detection signal to said blood analyte concentration.

3. The apparatus according to claim 2 wherein said converting of said detection circuit by said electronic circuitry comprises applying a correction factor to said detection signal.

4. The apparatus according to claim 3 wherein said electronic circuitry measures a transit time for each of said calibration and detection signals, and calculates said detection factor as a ratio of the measured transit times.

5. The apparatus of claim 1 further comprising a contact pad contacting the ultrasound transducer and the subject tissue.

6. The apparatus of claim 1 further comprising a display component connected to the electronic circuit.

7. The apparatus of claim 1 wherein the ultrasound signal has a frequency between about 2 MHz to about 20 MHz.

8. The apparatus according to claim 2 wherein said calibration signal has a frequency above about 10 MHz and said detection signal has a frequency below about 6 MHz.

9. The apparatus of claim 1 wherein the electronic circuitry further comprises:
a) a waveform generator connected to the transducer;
b) an ultrasonic signal discriminator connected to the transducer;
c) an analog-to-digital converter, and;
e) a central processing unit.

10. The apparatus of claim 1 further comprising an ultrasound reflective surface adapted to be positioned opposite from the ultrasound transducer when said transducer is positioned adjacent said subject tissue.

11. The apparatus of claim 10 wherein the reflective surface is metallic.

12. The apparatus of claim 1 further comprising an attachment device that attaches the apparatus to the subject.

13. The apparatus of claim 1 further comprising a removable data storage device, the data storage device adapted to connect to and disconnect from the electronic circuitry.

14. A method of measuring a blood constituent comprising:
a) emitting a calibration frequency signal into a subject tissue from a first position;
b) detecting a calibration echo signal from a reflection of the calibration frequency signal;
c) measuring a calibration transmittal time between emitting the calibration frequency signal and detecting the calibration echo signal;
d) emitting a detection frequency signal with a first amplitude into the subject tissue from the first position;
e) detecting an echo signal from the detection frequency signal and measuring a second amplitude of the detection frequency echo signal;
f) measuring a detection transmittal time between emitting the detection frequency signal and detecting the detection echo signal;
g) calculating a concentration of the blood constituent in the subject tissue from the calibration transmittal time, the detection transmittal time and an amplitude difference between the first and second detection frequency amplitudes.

15. The method of claim 14 wherein the calibration frequency signal and the detection frequency signal are ultrasonic frequency signals.

16. The method of claim 15 wherein the ultrasonic frequency is between about 2 MHz and about 20 MHz.

17. The method of claim 16 wherein the calibration frequency is higher than the detection frequency.

18. The method of claim 14 further comprising reflecting the calibration frequency and the detection frequency signals off a reflective surface.

19. The method of claim 14 further comprising accessing a look-up table to determine the concentration of the blood constituent.

20. The method of claim 14 wherein the blood constituent is glucose.

21. The method of claim 14 wherein the subject tissue is selected from the group consisting of an earlobe, an arm, a leg and a finger.

22. A non invasive detector for measuring a blood constituent the detector comprising:
an ultrasonic transducer adapted to be placed on a subject tissue;
electronic circuitry associated with the transducer, said circuitry comprising:
1. a driving means for driving said transducer to emit a calibration ultrasonic signal having a first frequency and a detection ultrasonic signal having a second frequency;
2. a signal detection means for receiving said calibration and said detection ultrasonic signals after said signals have passed through said subject tissue;
3. a signal processing means for:
(a) measuring said calibration signal and said detection signals transit time through said subject tissue;
(b) measuring and comparing a change between an amplitude of the detection ultrasonic signals before and after the signal has passed through said subject tissue; and
(c) calculating said measured blood constituent; and
4. a signal output means indicating said measured blood constituent.

23. The apparatus according to claim 22 wherein said signal processing means for calculating said blood constituent further comprise means for calculating a ratio of said signals transit times and for multiplying said amplitude change with said transit time ratio.

24. The apparatus according to claim 22 further comprising a contact pad contacting the ultrasonic transducer and the subject tissue.
